(19) United States
(12) Patent Application Publication
(10) Pub. No.: US 2024/0207824 A1
(43) Pub. Date: Jun. 27, 2024

(54) ALKYLENE OXIDE CATALYST THAT CAN BE MANUFACTURED RAPIDLY IN ONE STEP

(71) Applicant: Dow Global Technologies LLC, Midland, MI (US)

(72) Inventors: Karena Smoll, Houston, TX (US); Jasper Van Noyen, Wilrijk (BE); Jorge H. Pazmino, Pearland, TX (US); Vera P. Santos Castro, Terneuzen (NL); Mark H. McAdon, Midland, MI (US); Anny Liu, Pearland, TX (US)

(21) Appl. No.: 18/554,600

(22) PCT Filed: May 26, 2022
(86) PCT No.: PCT/US2022/031025

§ 371 Date: Oct. 9, 2023

Related U.S. Application Data
(60) Provisional application No. 63/222,122, filed on Jul. 15, 2021.

(51) Int. Cl. B01J 23/68; B01J 21/04; B01J 37/02; B01J 37/08; C07D 301/10

(52) U.S. Cl. CPC B01J 23/688; B01J 21/04; B01J 37/0201; B01J 37/08; C07D 301/10

(57) ABSTRACT

A supported silver catalyst and use thereof in a process for producing an olefin oxide, such as ethylene oxide, by the direct oxidation of an alkylene with oxygen or an oxygen-containing gas, wherein the catalyst provides good catalyst activity and/or efficiency despite loading levels of silver in the range of 16 to 25%.

### BACKGROUND OF THE INVENTION

This invention pertains to a supported silver catalyst, its manufacture, and its use in the production of an alkylene oxide, particularly, ethylene oxide, directly from oxygen and an olefin, such as ethylene.

Alkylene oxides are known for a multiplicity of utilities. Ethylene oxide, for example, is used to produce ethylene glycol, which is used in preparing polyester fibers and resins, nonionic surfactants, glycol ethers, ethanolamines, and polyethylene polyether polyols. Propylene oxide is used to produce propylene glycol and polypropylene polyether polyols, which are used in polyurethane polymer applications.

The manufacture of ethylene oxide by the direct reaction of ethylene with oxygen or an oxygen-containing gas in the presence of a silver catalyst is an old and well-developed art. An outline of the history of direct ethylene oxidation can be found in U.S. Pat. No. 4,916,243. This patent, more particularly, describes a catalyst comprising silver deposited on an alumina macroporous support, further comprising cesium and at least one other alkali metal selected from the group consisting of lithium, sodium, potassium, and rubidium, such that the combination of cesium and other alkali metals exhibits a synergistic promoting effect on the oxidation process.

A supported silver catalyst for alkylene oxide manufacture should have acceptable activity, efficiency, and stability. The “activity” of a catalyst can be quantified in a number of ways, one being the mole percent of alkylene oxide contained in the outlet stream of the reactor relative to that in the inlet stream (the mole percent of alkylene oxide in the inlet stream typically, but not necessarily, approaches zero percent) while the reactor temperature is maintained substantially constant; and another being the temperature required to maintain a given rate of alkylene oxide production. In many instances, activity is measured over a period of time in terms of the mole percent of alkylene oxide produced at a specified constant temperature. Alternatively, activity may be measured as a function of the temperature required to sustain production of a specified constant mole percent of alkylene oxide, such as ethylene oxide. The “efficiency” of the oxidation, which is synonymous with “selectivity,” refers to the total amount, in molar percent, of converted or reacted olefin that forms a particular product. For example, the “selectivity to alkylene oxide” refers to the mole percent of converted or reacted olefin that forms alkylene oxide. One measure of the useful life of a catalyst is the length of time that reactants can be passed through the reaction system during which time acceptable productivity is obtained in light of all relevant factors. “Deactivation”, as used herein, refers to a permanent loss of activity and/or efficiency, that is, a decrease in activity and/or efficiency that cannot be recovered. Generally, deactivation tends to proceed more rapidly when higher reactor temperatures are employed. The “stability” of a catalyst is inversely proportional to the rate of deactivation. Lower rates of deactivation are generally desirable.

In recent years, improvements in activity, efficiency, and stability of alkylene oxide catalysts have been achieved with the use of modified alumina carriers prepared, preferably, from high-purity alpha-alumina of greater than 95 weight percent compositional purity. WO-A1-2005/023417, for example, discloses modification of high-purity preformed alpha-alumina carriers by impregnating the carrier with an alkali metal hydroxide, such as sodium hydroxide, and thereafter washing the carrier to remove unbound or excess alkali. Thereafter, the modified carrier is impregnated with silver and cesium, and optionally, additional promoters, such as rhenium, manganese, and/or other alkali metals. Likewise, WO-A1-2005/039757 discloses modification of high-purity alpha-alumina carriers with zirconium silicate (zircon), after which the zircon-modified alumina is impregnated with silver and one or more promoting cations or anions. The carriers and catalysts derived from these modification processes typically do not contain binders, such as clays. Binders tend to introduce undesirable quantities of extraneous metals, so avoidance of binders is desirable.

Other references, such as WO-A1-2007/123932, describe high efficiency catalysts modified for better performance in the event of reactor upsets.

These reported catalysts typically rely on a relatively high loading of silver, typically greater than 30 or even 35 percent by weight. To achieve such high levels of silver, typically the manufacturing process requires multiple impregnation steps, increasing the costs of producing the catalysts, and decreasing the annual production capacity of a catalyst manufacturing plant. Moreover, particularly with larger ethylene oxide production plants, the total amount of silver required can be a significant capital demand, especially considering the rising price of silver. Accordingly, it would be desirable to have high efficiency silver catalysts for the production of ethylene oxide that have a silver content of less than about 25 percent by weight, particularly catalysts which can be prepared using a single step of silver deposition.

### SUMMARY OF THE INVENTION

The present inventors selected and screened several hundred promoter compositions and identified trends in catalyst activity and catalyst selectivity using regression models to fit the experimental data. It was discovered that through the use of carefully tailored amounts of certain alkali and oxyanion promoters, lower amounts of silver can be used without unduly sacrificing either the activity or selectivity of such catalysts. Relative to catalysts with silver content of about 33 percent by weight, the invented catalysts with silver less than about 25 percent by weight actually have higher levels of promoters. This was a surprising result because increasing promoter levels by linear scaling tends to decrease the catalyst activity (see U.S. Pat. Nos. 9,649,621B2 and 9,908,861B2). For catalysts with less than 25 percent silver, it was expected that promoter levels would need to be decreased in order to compensate for the expected activity penalty on decreasing the silver content from about 33 percent by weight to less than 25 percent by weight. Therefore, in one aspect, the invention is a supported silver catalyst prepared on an alumina-containing carrier. The carrier is a high-purity alumina carrier, having greater than about 80 weight percent alpha-alumina and less than about 30 parts per million acid-leachable alkali metals selected from lithium, sodium, potassium, and mixtures thereof, by weight, the weight percent of the alumina and the concentration of the acid-leachable alkali metals being calculated on the weight of the carrier. Onto this carrier is deposited: (A) silver in an amount of from 16 to 25 percent by weight of the catalyst; and (B) a solid promoter package comprising cesium, sulfate, rhenium, sodium and optionally lithium. Preferably manganese in an amount of 20 to 300 ppm is also deposited on the catalyst. For promoters other than manganese, the loadings are expressed in units of millimoles of promoter per kilogram of catalyst, and the loadings are scaled by a factor of Q, where Q is a unitless or dimensionless scaling factor equal to the surface area of the alumina-containing carrier prior to deposition of silver and promoters in units of square meters per gram divided by one square meter per gram.

The amounts of the components in the solid promoter package deposited on the catalyst are: C_{Cs}/Q in a range of from 3.1 to 8.7 mmol/kg catalyst; C_{Na}/Q in a range of 0.5 to 7.5 mmol/kg catalyst; C_{S}/Q in a range of 0.3 to 3.2 mmol/kg catalyst; C_{Re}/Q in a range of 2.4 to 6.9 mmol/kg catalyst; and C_{Li}/Q in a range of 0 to 35 mmol/kg catalyst, where C_{Cs}, C_{Li}, C_{Na}, C_{S}, and C_{Re} are the amounts of cesium, lithium, sodium, sulfate, and rhenium, respectively, deposited on the carrier expressed in units of mmol promoter per kg of catalyst.

Furthermore, these amounts are balanced in such a manner that F1/Q is in a range of from 0.3 to 5.2 mmol/kg catalyst; and F2/Q is in a range of from −5.1 to 6.3 mmol/kg catalyst, where F1 and F2 are linear combinations of the promoter deposited loadings as defined by the following equations:

$$F 1 = C {c}_{s} + 0.032 \cdot {C}_{L i} + 0.47 \cdot {C}_{N a} - ( 0.72 \cdot Cs + 0.94 \cdot {C}_{R e} ) ; \qquad eq . 1$$

$$F 2 = C {c}_{s} - 0.24 \cdot {C}_{L i} - 0.27 \cdot {C}_{N a} + 0.3 \cdot Cs . \qquad eq . 2$$

The aforementioned catalyst of this invention exhibits utility in a continuous process for manufacturing an alkylene oxide directly from an olefin and oxygen or an oxygen-containing gas. Advantageously, the catalyst of this invention exhibits comparable levels of activity and/or efficiency, as compared with previously reported catalysts having higher levels of silver.

### BRIEF DESCRIPTION OF THE DRAWING

FIG. 1 is a graph depicting the selectivity and the activity of catalysts 11-45.

FIG. 2 is a graph depicting the selectivity and the activity of catalysts 46-57.

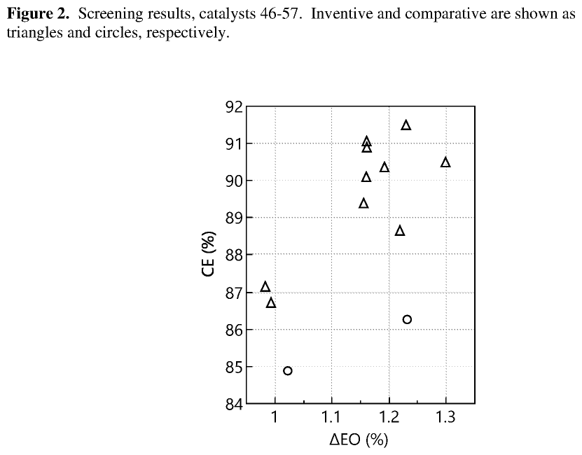

### DETAILED DESCRIPTION OF THE INVENTION

The invention described herein provides for a novel supported silver catalyst which finds utility in the direct oxidation of an alkylene (olefin), such as ethylene, with oxygen or an oxygen-containing gas to form an alkylene oxide, such as ethylene oxide, and which has a silver content of 25 percent by weight or less, preferably 24 percent or less, 23 percent or less, or even 22 percent or less. It has been found that good results in terms of ethylene oxide production can be obtained when the silver content is at least 16 percent, preferably 17 percent, 18 percent or even 19 percent by weight. More specifically, the supported silver catalyst of this invention comprises an alumina carrier comprising less than about 30 parts per million, preferably less than 25 parts per million acid-leachable alkali metals by weight, the concentration of the alkali metals being calculated on the weight of the carrier, wherein the alkali metals are selected from lithium, sodium, potassium, and mixtures thereof.

It is preferred that the supported silver catalyst comprises an alumina carrier comprising at least about 80 percent or greater, preferably at least 90 percent, 95 percent or even 98 percent alpha-alumina.

It is preferred that the carrier have a surface area as expressed in units of square meters of surface area per gram of carrier, of not less than 0.7 m²/g, 0.8 m²/g, 0.9 m²/g, or 1.0 m²/g. In general, the higher the surface area the better, but in some embodiments, the carrier surface area is no greater than 1.5 m²/g, or 1.4 m²/g, or 1.3 m²/g. Suitable carriers can be made pursuant to procedures known in the art, such as those described in WO2005/039757.

To this carrier, the following is deposited thereon: (A) silver; and (B) an additional solid promoter package comprising cesium, sodium, sulfate, rhenium, and optionally lithium. Preferably manganese is also deposited as a promoter.

The preferred level (i.e., amount) of promoters will depend in part on the surface area of the carrier, as expressed in units of square meters of surface area per gram of carrier. The surface area of the carrier is measured by nitrogen BET and the pore volume and median pore diameter are measured by mercury porosimetry, as is generally known in the art, for example as shown in WO2007/123932.

The amount of manganese, when present, should be in a range of 20 to 300 ppm, by weight of the catalyst. Preferably manganese is present in an amount of at least 50, 70, or 90 ppm up to 250, 200, or 150 ppm.

It is preferred that cesium be present in an amount of C_{Cs}/Q from 3.1, 3.7, 4.2, or 4.7 up to 8.7, 8.4, 7.8 or 7.3 mmol per kg of catalyst, where Q is unitless/dimensionless and equal to the surface area of the alumina-containing carrier prior to deposition of silver and promoters expressed in units of square meters per gram divided by one square meters per gram, and C_{Cs} is the amount of cesium deposited on the carrier expressed in units of mmol promoter per kg of catalyst.

It is preferred that deposited sodium be present in an amount of C_{Na}/Q from 0.5, 1.2, 1.8 or 2.5 up to 7.5, 7.0, 6.5, or 6.0 mmol/kg catalyst and C_{Na} is the amount of sodium deposited on the carrier expressed in units of mmol promoter per kg of catalyst.

It is preferred that sulfate be present in an amount of C_{S}/Q from 0.3, 0.5, or 0.7 up to 3.2, 2.7, or 2.2 mmol/kg catalyst where C_{S} is the amount of sulfate deposited on the carrier expressed in units of mmol promoter per kg of catalyst.

It is preferred that rhenium be present in an amount of C_{Re}/Q from 2.4, 2.8, or 3.3 up to 6.9, 6.4, or 6.0 mmol/kg catalyst where C_{Re} is the amount of rhenium deposited on the carrier expressed in units of mmol promoter per kg of catalyst.

While optional, it is preferred that deposited lithium be present in an amount of C_{Li}/Q from 0, 3, 6, or 10, up to 35, 30, or 26 mmol/kg catalyst where C_{Li} is the amount of lithium deposited on the carrier expressed in units of mmol promoter per kg of catalyst.

It should be understood that the above amounts of the various promoters are the amounts to be deposited on the carrier and do not include any amounts that may initially be present in the alumina-containing carrier, for example, as impurities.

As will be appreciated by one of ordinary skill in the art, the above ranges of promoters are generally higher that what has previously been reported. It is surprising that activity is maintained under these conditions as publications such as U.S. Pat. Nos. 9,649,621(B2) and 9,908,861(B2) have suggested that higher amounts of alkali promoters generally lead to poorer activity in catalysts.

It is preferred that the promoters are balanced in relation to one another. It is preferred that F1/Q is in a range of from 0.3, 0.6, 1.0, 1.5, or 1.9 to 5.2, 4.9, 4.5, 4.1, or 3.7 mmol/kg catalyst, and it is preferred that F2/Q is in a range of from −5.1, −4.4, −3.6, −2.5, or −1.8 to 6.3, 5.6, 4.7, 3.5, or 2.7 mmol/kg catalyst, where F1 and F2 are defined in equations 1 and 2, respectively.

The promoters can be added in any convenient form, for example cesium hydroxide, cesium acetate, lithium acetate, ammonium sulfate, ammonium perrhenate, sodium acetate and manganese nitrate. It may be desirable to premix the manganese promoter (when present) with ethylenediaminetetraacetic acid (EDTA) prior to addition to the silver impregnating solution.

In another aspect, this invention provides for a continuous process for the production of alkylene oxide comprising contacting in a vapor phase an alkylene, preferably ethylene, with oxygen or an oxygen-containing gas in the presence of a supported silver catalyst, the catalyst comprising any of the compositions identified hereinbefore; and the contacting being conducted under process conditions sufficient to produce the alkylene oxide.

Alkylenes (olefins) employed in the process of this invention are preferably characterized by the following structural formula I:

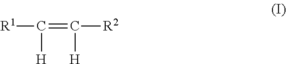

wherein R¹ and R² are each individually selected from hydrogen and lower monovalent alkyl radicals, preferably, C₁₋₆ alkyl radicals, such as, methyl, ethyl, propyl, butyl, and higher homologues up to six carbon atoms. Preferably, R¹ and R² are each individually selected from hydrogen, methyl, and ethyl. More preferably, each R¹ and R² is hydrogen, and the preferred olefin is ethylene. The corresponding alkylene oxides produced in the process of this invention are preferably characterized by the following structural formula II:

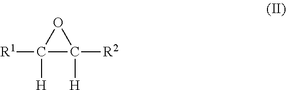

wherein R¹ and R² are identified hereinbefore in connection with the reactant olefin. Most preferably, the alkylene oxide is ethylene oxide.

As known from the prior art, oxygen may be provided to the process as pure molecular oxygen, or alternatively, as an oxygen-containing gas, wherein the gas further contains one or more gaseous components, for example, gaseous diluents, such as nitrogen, helium, methane, and argon, which are essentially inert with respect to the oxidation process. A suitable oxygen-containing gas, for example, is air. Additionally, the oxygen-containing gas may contain one or more of the following gaseous components including water, carbon dioxide, and various gaseous promoters and/or gaseous by-product inhibitors as discussed hereinafter.

The relative volumetric ratio of alkylene to oxygen in the feed gas may range in accordance with any of such known conventional values. Typically, the volumetric ratio of alkylene to oxygen in the feed is subject to flammability limits, as is well known in the art, and may vary from about 2/1 to about 10/1. Likewise, the quantity of inert gases, diluents, or other gaseous components, such as water, carbon dioxide, gaseous promoters, and gaseous by-product inhibitors, may vary in accordance with known conventional ranges as found in the art.

The catalyst carrier employed in practicing the invention may be selected from any of the known alumina carriers, modified or unmodified, that contain high-purity alumina, specifically, a high-purity alumina compositionally comprising greater than about 80, preferably, greater than about 90, more preferably, greater than about 95, and most preferably, greater than about 98 weight percent alumina. The compositional balance typically comprises any of zirconium silicate (zircon), other refractory silicates, silica, or other metal oxides. In terms of phase composition, the alumina preferably comprises alpha-phase alumina (alpha-alumina), and more preferably, greater than about 99 percent alpha-phase alumina (alpha-alumina). As a necessary condition, the high-purity alumina carrier should contain less than about 30 ppm, preferably, less than about 25 ppm, and more preferably, less than about 20 ppm, acid-leachable alkali metals by weight, the concentration of the alkali metals being calculated on the weight of the carrier, wherein the alkali metals are selected from lithium, sodium, potassium, and mixtures thereof. Preferably, the high-purity alumina carrier contains less than about 30 ppm, more preferably, less than about 25 ppm, by weight, acid-leachable sodium.

In some embodiments, the alumina carrier also contains zirconium silicate (zircon), more preferably, in any amount up to about 4, 3, or 2 weight percent, calculated on the weight of the carrier.

No limits are placed on the method in which the low levels of alkali metals (Li, Na, K) forming a compositional part of the high-purity alumina carrier are incorporated into the carrier, if the alkalis are present at all. Typically, these alkali metals are introduced into the carrier during its synthesis, for example, as impurities in one or more of the raw materials, or as contaminants in the firing environment; but other methods of achieving such levels of these alkali metals may be possible. What is important is that the catalyst of this invention is prepared starting from a pre-formed high-purity alumina carrier having less than about 30 ppm acid-leachable alkali metals selected from lithium, sodium, potassium, and mixtures thereof. Thereafter, the pre-formed high-purity alumina carrier is treated so as to intentionally deposit, i.e., add thereto, silver and a solid promoter package comprising cesium, sodium, sulfate, rhenium, and optionally lithium and/or manganese.

Representative examples of materials which can be employed as the high-purity alumina according to the present invention include such carriers as manufactured by Süd Chemie, Inc., Louisville, Ky., and Saint-Gobain NorPro Corp., Akron, OH. Other suppliers are also available.

Suitable shapes for the high-purity alumina carrier include any of the wide variety of shapes known for such carriers or supports, including, pills, chunks, tablets, pieces, pellets, rings, spheres, wagon wheels, toroids having star shaped inner and/or outer surfaces, and the like, of a size suitable for employment in fixed bed reactors. Conventional commercial fixed bed ethylene oxide reactors are typically in the form of a plurality of parallel elongated tubes (in a suitable shell) about 1 to 3 inches (2.5 to 7.5 cm) outer diameter and about 15 to 45 feet (4.5 to 13.5 m) long filled with catalyst. In such fixed bed reactors, it is desirable to employ a carrier formed into a rounded shape, such as, for example, spheres, pellets, rings, tablets, and the like, having diameters from about 0.1 inch (0.25 cm) to about 0.8 inch (2 cm).

There are many well-known methods of preparing alumina carriers suitable for use in alkylene oxide catalysts of the present invention. Some of such methods are described, for example, in international patent application publication WO-A1-2005/039757; and in U.S. Pat. Nos. 4,994,587; 4,994,588; and 5,504,053, incorporated herein by reference. Preferably, an alumina support of at least 90 percent purity having desirable properties (such as, desirable morphology, surface area, pore volume, and/or pore size distribution) can be prepared by compounding (mixing) the raw material, extruding, drying, and high-temperature calcining. In this case, the raw material usually includes one or more alpha-alumina powder(s) with different properties, and optionally, a material that may be added to provide physical strength, and optionally, a burnout material (usually an organic compound) used to provide desired porosity after removal by calcination, provided that the binder and burnout material do not add a quantity of alkali metal (Li, Na, K) to the carrier beyond the required upper limit of less than about 30 ppm by weight. The levels of impurities in the finished carrier are largely determined by the purity of the raw materials used and their degree of volatilization during the calcination step. Common impurities include silica, alkali and/or alkaline earth metal oxides, and trace amounts of metal and/or non-metal containing additives.

Another known method for preparing high-purity alpha-alumina having suitable properties comprises mixing zirconium silicate with boehmite alumina (AlOOH) and/or gamma-alumina, peptizing the boehmite alumina and/or gamma-alumina in an acidic mixture containing halide anions (preferably fluoride anions) to provide halogenated alumina; forming (for example, by extruding or pressing) the peptized halogenated alumina to provide formed peptized halogenated alumina; drying the formed peptized halogenated alumina to provide dried formed alumina; and calcining the dried formed alumina to provide pills of alpha-alumina carrier. Where alpha-alumina carrier is used that has been prepared as described in this paragraph, it is important that the alumina, which has been peptized with an acidic mixture containing halide anions, be calcined before deposition of silver or promoting metals, because the halide is necessary for forming desirable platelets of alpha-alumina, as noted hereinbelow.

The high-purity alpha-alumina carrier for use in the present invention preferably has a specific surface area of at least about 0.5 m²/g, and more preferably, at least about 0.7 m²/g. The surface area is typically no greater than about 10 m²/g, and often no greater than about 5 m²/g, 2 m²/g or even 1.5 m²/g. The high-purity alumina carrier preferably has a pore volume of at least about 0.5 cm³/g, and more preferably, from about 0.5 cm³/g to about 2.0 cm³/g; and a median pore diameter from about 1 to about 50 microns. Preferably, the high-purity alumina has a crush strength of greater than about 12 pounds. The high-purity alpha-alumina preferably includes particles each of which has at least one substantially flat major surface having a lamellate or platelet morphology which approximates the shape of a hexagonal plate (some particles having two or more flat surfaces), at least 50 percent of which (by number) have a major dimension of less than about 50 microns.

Catalysts of this invention for the production of alkylene oxide, for example, ethylene oxide or propylene oxide, may be prepared with the aforementioned high-purity alpha-alumina, by impregnating the carrier with a solution of one or more silver compounds, as is well known in the art. The solid promoter package may be impregnated simultaneously with the silver impregnation, or before the silver impregnation, or after the silver impregnation. It is preferred that the impregnation of the silver and promoters is carried out simultaneously.

The art discloses the concept of “promoters,” that is, materials which, when present in combination with the catalytic silver, benefit one or more aspects of catalyst performance or otherwise act to promote the catalyst's ability to make a desired alkylene oxide product, preferably, ethylene oxide or propylene oxide. Such promoters in themselves are generally not considered catalytic materials; however, the presence of such promoters in the catalyst has been shown to contribute to one or more beneficial effects on the catalyst performance, for example, enhancing the rate or amount of production of desired product (for example, by enhancing activity and/or efficiency), reducing the temperature required to achieve a suitable rate of reaction, and/or reducing the rates or amounts of undesired by-product reactions. Competing reactions occur simultaneously in the reactor, and a critical factor in determining the effectiveness of the overall process is the measure of control one has over these competing reactions. A material which is termed a promoter of a desired reaction can be an inhibitor of another reaction, for example a combustion reaction. What is significant is that the effect of the promoter on the overall reaction is favorable to the efficient production of the desired product, in this case alkylene oxide, and more preferably, ethylene oxide.

It has now been discovered that when the concentrations of the various promoter components are carefully controlled, use of the catalyst to produce alkylene oxide, and in particular ethylene oxide can result in desired activity and selectivity levels despite lower amounts of silver deposited on the catalyst. The concentrations of components of the solid promoter package (cesium, sodium, sulfate, rhenium, and optionally lithium and/or manganese) as stated above, are provided in a promoting amount. In this context, the term “promoting amount” means an amount of the promoter that provides an improvement in one or more of the catalytic properties of that catalyst when compared to a comparative or baseline catalyst containing the same amounts of same components, however, without said promoting component, and when compared under the same (controlled) process conditions. Examples of catalytic properties include, inter alia, resilience, operability (resistance to run-away), activity, conversion (e.g., conversion of alkene), efficiency (selectivity), stability, and yield. Preferably, the promoters are provided in a “synergistic combination.” The term “synergistic combination” refers to the selection of promoters in appropriate quantities, which are capable of achieving an efficiency greater than the value obtainable under similar operating conditions from respective catalysts having been prepared from individual components of the solid promoter package. U.S. Pat. No. 4,913,243, incorporated herein by reference, teaches a silver-supported catalyst containing a synergistic combination of cesium and at least one other alkali metal selected from the group consisting of lithium, sodium, potassium, and rubidium. Such patent describes an efficiency equation that may be useful in identifying a synergistic combination of the cesium and other alkali metal(s); but said efficiency equation represents only one method, not the only method, of characterizing synergistic combinations.

Well known methods can be employed to analyze for the amounts of silver, and the individual components of the solid promoter package deposited onto the alumina carrier. The skilled artisan may employ, for example, material balances to determine the amounts of any of these deposited components. As an example, if the alumina carrier is weighed prior to and after deposition of silver and an alkali metal-containing compound, then the difference in the two weights will be equal to the amount of silver and the alkali metal-containing compound deposited onto the carrier, from which the amount of deposited alkali metal can be calculated. Additionally, the amount of the deposited silver and alkali metal-containing compound can be calculated based upon the ratio of the concentration of silver and alkali metal-containing compounds in the impregnation solutions and the weight picked up from the impregnation solutions. Alternatively, any suitable analytical technique for determining elemental composition, such as inductively coupled plasma (ICP) or X-ray fluorescence (XRF) spectrometry, may be employed to determine the amounts of deposited components. As an example, an alumina carrier can be analyzed by XRF to determine the amount of cesium present in the carrier. After impregnation with a cesium-containing compound, the impregnated carrier may be analyzed by XRF again to determine the total amount of cesium present in and deposited onto the carrier. The difference in the measurements reflects the amount of cesium deposited onto the carrier.

Besides the solid promoter package described hereinabove, gaseous promoters may, if desired, be employed with the catalyst of this invention. Gaseous promoters are gas-phase compounds and/or mixtures thereof which are introduced to a reactor for the production of alkylene oxide (preferably, ethylene oxide) with the vapor-phase reactants, such as ethylene and oxygen. Such promoters, also called modifiers, inhibitors, or enhancers, further enhance the performance of a given catalyst, working in conjunction with or in addition to the solid promoters. One or more chlorine-containing components are typically employed as gaseous promoters, as is well known in the art. Other halide-containing components may also be used to produce a similar effect.

The solid promoter package is generally added as chemical compounds to the catalyst prior to its use. As used herein, the term “compound” refers to the combination of a particular element with one or more different elements by surface and/or chemical bonding, such as ionic and/or covalent and/or coordinate bonding. The term “ionic” or “ion” refers to an electrically charged chemical moiety; “cationic” or “cation” being positive and “anionic” or “anion” being negative. The term “oxyanionic” or “oxyanion” refers to a negatively charged moiety containing at least one oxygen atom in combination with another element. An oxyanion is thus an oxygen-containing anion. It is understood that ions do not exist in vacuo but are found in combination with charge-balancing counter ions when added as a compound to the catalyst. Once in the catalyst, the form of the promoter is not always known, and the promoter may be present without the counter ion added during the preparation of the catalyst. For example, a catalyst made with cesium hydroxide may be analyzed to contain cesium but not hydroxide in the finished catalyst. Likewise, compounds such as alkali metal oxide, for example cesium oxide, while not being ionic, may convert to ionic compounds during catalyst preparation or in use. For the sake of ease of understanding, the solid promoters will be referred to in terms of cations and anions regardless of their form in the catalyst as prepared and/or under reaction conditions.

Generally, the carrier is impregnated with a catalytic amount of silver, which is any amount of silver capable of catalyzing the direct oxidation of the alkylene with oxygen or an oxygen-containing gas to the corresponding alkylene oxide. In making such a catalyst, the carrier is typically impregnated (one or more times) with one or more silver compound solutions sufficient to allow the silver to be supported on the carrier in the desired range of from about sixteen to 1 no more than about twenty-five percent by weight, based on the weight of the catalyst. Most preferably the carrier is impregnated one time with a solution comprising a silver compound to obtain the desired silver content.

The silver solution used to impregnate the carrier is preferably comprised of a silver compound in a solvent or complexing/solubilizing agent such as the silver solutions disclosed in the art. The particular silver compound employed may be chosen, for example, from among silver complexes, silver nitrate, silver oxide, or silver carboxylates, such as silver acetate, oxalate, citrate, phthalate, lactate, propionate, butyrate, and higher fatty acid salts. Silver oxide complexed with amines is another preferred form of silver for use in the present invention.

A wide variety of solvents or complexing/solubilizing agents may be employed to solubilize silver to the desired concentration in the impregnating medium. Among those disclosed as being suitable for this purpose are lactic acid; ammonia; alcohols, such as ethylene glycol; and amines and aqueous mixtures of amines.

For example, silver oxide (Ag₂O) can advantageously be dissolved in a solution of oxalic acid and ethylenediamine such that the resulting impregnation solution comprises approximately 26% silver oxide, 18% oxalic acid dihydrate, 17% ethylenediamine, 6% monoethanolamine and 31% water.

Vacuum impregnation of such a solution onto a carrier of approximately 0.7 cm³/g porosity typically results in a catalyst containing approximately 20 percent by weight of silver based on the entire weight of the catalyst. Previously, when it was desired to obtain a catalyst having a silver loading of greater than about 25 or 30 percent, and more, it would generally be necessary to subject the carrier to at least two or more sequential impregnations of silver, with or without promoters, until the desired amount of silver is deposited on the carrier, whereas supported catalyst of the present invention may be able to be produced using a single step of impregnation of silver, greatly simplifying the process to produce the catalyst.

Although silver particle size in the finished catalyst is important, the range is not narrow. A suitable silver particle size can be in the range of from about 10 to about 10,000 angstroms in diameter. A preferred silver particle size ranges from greater than about 100 to less than about 5,000 angstroms in diameter. It is desirable that the silver and the various components of the solid promoter package be relatively uniformly dispersed on the alumina carrier.

A preferred procedure for depositing silver catalytic material and the solid promoter package comprises: (1) impregnating a porous alumina carrier according to the present invention with a solution comprising a solvent or solubilizing agent, silver complex and the solid promoter package and (2) thereafter treating the impregnated carrier to convert the silver salt to silver metal and effect deposition of silver and the promoter(s) onto the exterior and interior pore surfaces of the carrier. Silver and promoter depositions are generally accomplished by heating the carrier at elevated temperatures to evaporate the liquid within the carrier and effect deposition of the silver and promoters onto the interior and exterior carrier surfaces.

Alternatively, a coating of silver, and the solid promoter package may be formed on the carrier from an emulsion or slurry containing the metal components followed by heating the carrier as described hereinabove. However, impregnation of the carrier is generally the preferred technique for silver deposition, because it utilizes silver more efficiently than coating procedures, the latter being generally unable to effect substantial silver deposition onto the interior surfaces of the carrier. In addition, coated catalysts are more susceptible to silver loss by mechanical abrasion.

As with the silver deposition, soluble salts of components of the solid promoter package may be dissolved in one or more solvents and/or solubilizing agents and deposited, preferably by impregnation, onto the carrier. The sequence of impregnating or depositing the surfaces of the carrier with silver and the components of the solid promoter package may vary. Thus, impregnation and deposition of silver, cesium, sodium, sulfate, rhenium, and optionally lithium and/or manganese may be effected coincidentally or sequentially, for example, cesium and sodium may be deposited prior to, during, or subsequent to silver deposition to the carrier. The individual components of the solid promoter package may be deposited together or sequentially. For example, silver may be deposited first followed by the coincidental or sequential deposition of cesium, lithium (if used), sulfate, and rhenium (or combinations thereof); or alternatively, cesium may be deposited first followed by coincidental or sequential deposition of silver and lithium (if used), sulfate and rhenium; or alternatively, if used, lithium may be deposited first followed by coincidental or sequential deposition of silver and cesium, sulfate and rhenium, etc. If two or more impregnations are employed, the impregnated carrier is typically dried, or calcined and/or roasted between each successive impregnation to ensure deposition of the metals onto the carrier.

Thereafter, the carrier, now impregnated with silver, and the solid promoter package comprising cesium, sodium, sulfate, rhenium and optionally lithium and/or manganese, is calcined or roasted under air at a temperature ranging from about 200° C. to about 600° C. and at atmospheric pressure for a time ranging from about 0.01 to about 12 hours. Temperatures of 475° C. to 525° C. for periods of 5 to 20 minutes are generally preferred. Alternatively, the calcination can be carried out in two or more different steps, with initial steps being conducted at generally lower temperatures.

The rhenium component can be provided in various forms, for example, as the metal, as a covalent compound, as a cation, or as an anion. The rhenium species that provides the enhanced efficiency and/or activity is not certain and may be the component added or that generated either during preparation of the catalyst or during use as a catalyst. Examples of rhenium compounds include the rhenium salts such as rhenium halides, the rhenium oxyhalides, the rhenates, the perrhenates, the oxides, and the acids of rhenium. However, the alkali metal perrhenates, ammonium perrhenate, alkaline earth metal perrhenates, silver perrhenates, other perrhenates, and rhenium heptoxide can also be suitably utilized, provided that in the case of the alkali metal perrhenates, the quantities of alkali metals (Cs and/or Rb; and Na and/or K) therein are taken into account when assessing the total of these cations deposited onto the carrier. Rhenium heptoxide, Re₂O₇, when dissolved in water, hydrolyzes to perrhenic acid, HReO₄, or hydrogen perrhenate. Thus, for purposes of this specification, rhenium heptoxide can be considered to be a perrhenate, that is, monoanionic ReO₄.

Another class of preferred promoters and catalyst stabilizers, which may be employed with the present invention, includes manganese components. In many instances, manganese components can enhance the activity, efficiency, and/or stability of catalysts. The manganese species that provides the enhanced activity, efficiency, and/or stability is not certain and may be the component added or that generated either during catalyst preparation or during use as a catalyst. Manganese components include, but are not limited to, manganese acetate, manganese ammonium sulfate, manganese citrate, manganese dithionate, manganese oxalate, manganous nitrate, manganous sulfate, and manganate anion, for example permanganate anion, and mixtures thereof. To stabilize the manganese component in certain impregnating solutions, it may be necessary to add a chelating compound, such as, ethylenediaminetetraacetic acid (EDTA) or a suitable salt thereof.

The promoting effect(s) provided by the solid promoter package and optional gas phase promoters can be affected by a number of variables, for example, reaction conditions, catalyst preparation techniques, surface area and pore structure, and surface chemical properties of the support, the silver, and the concentrations of the promoters present in the catalyst.

The present invention is applicable to epoxidation reactions in any suitable reactor, for example, fixed bed reactors, continuous stirred tank reactors (CSTR), and fluid bed reactors, a wide variety of which are well known to those skilled in the art and need not be described in detail herein. The desirability of recycling unreacted feed, or employing a single-pass system, or using successive reactions to increase ethylene conversion by employing reactors in series arrangement can also be readily determined by those skilled in the art. The particular mode of operation selected is usually dictated by process economics. Conversion of olefin (alkylene), preferably ethylene, to olefin oxide, preferably ethylene oxide, can be carried out, for example, by continuously introducing a feed stream containing alkylene (e.g., ethylene) and oxygen or an oxygen-containing gas to a catalyst-containing reactor at a temperature of from about 200° C. to about 300° C., and a pressure which may vary within the range of from about 5 atmospheres (506 kPa) to about 30 atmospheres (3.0 MPa), depending upon the mass velocity and productivity desired. Residence times in large-scale reactors are generally on the order of about 0.1 to about 5 seconds. Oxygen may be supplied to the reaction in an oxygen-containing stream, such as, air or as commercial oxygen, or as oxygen-enriched air. The resulting alkylene oxide, preferably, ethylene oxide, is separated and recovered from the reaction products using conventional methods.

Prior to production of alkylene oxide, it is generally desirable to activate or break in the catalyst, as is generally known by those skilled in the art. One suitable activation protocol is to expose the supported catalyst at 245° C. at near-optimal ethyl chloride concentration for two to five days to quickly achieve optimum performance.

The catalysts disclosed herein can be used under widely varying process conditions, as is well known by those skilled in the art.

The following examples are set forth for the purpose of illustrating the invention; but these examples are not intended to limit the invention in any manner. One skilled in the art will recognize a variety of substitutions and modifications of the examples that will fall within the scope of the invention.

### Examples

A series of high purity alpha-alumina carriers having hollow shaped geometries, and having greater than about 80 weight percent alpha-alumina and less than about 30 parts per million acid-leachable alkali metals (particularly lithium, sodium and potassium) by weight, the weight percent of the alumina and the concentration of the acid-leachable alkali metals being calculated on the weight of the carrier, are obtained from Saint-Gobain NorPro. Table 1 below shows the properties for Carriers A-F.

TABLE 1
Carrier Properties

| | | surface | pore | packing | zircon |
|---|---|---|---|---|---|
| | carrier | area | volume | density | (parts per |
| carrier | shape | (m²/g) | (cm³/g) | (kg/m³) | thousand) |
| A | penta-ring | 1.16 | 0.70 | 524 | 21 |
| B | penta-ring | 1.12 | 0.68 | 521 | 22 |
| C | penta-ring | 1.20 | 0.69 | 524 | 22 |
| D | penta-ring | 1.15 | 0.68 | 532 | 23 |
| E | mono-ring | 1.14 | 0.61 | 604 | 21 |
| F | mono-ring | 1.05 | 0.66 | 561 | 0 |
| G | penta-ring | 1.16 | 0.66 | 533 | 22 |
| H | penta-ring | 1.28 | 0.69 | 533 | 18 |

### Silver Compound Solution

The silver impregnation solution is prepared in accordance with the procedure described in US 2009/0177000 A1 and contains, by weight, approximately 27% silver oxide, 18% oxalic acid dihydrate, 17% ethylenediamine, 6% monoethanolamine, and 31% water. To this pre-prepared silver solution, the individual promoter solutions are added in quantities that are pre-calculated to create the desired promoter composition on the finished catalysts.

### Promoter Solutions, Synthesis by Vacuum Impregnation

Manganese nitrate (Mn(NO₃)₂), diammonium ethylenediaminetetraacetic acid ((NH₄)₂H₂(EDTA)), cesium hydroxide (CsOH), lithium acetate (LiOCOCH₃), and ammonium sulfate ((NH₄)₂SO₄) are used as pre-made solutions. The manganese and EDTA solutions are pre-mixed prior to addition into pre-prepared silver solution. CsOH solution is typically diluted with deionized water to the desired cesium concentration before use. Sodium acetate (NaOCOCH₃) promoter solution is made by dissolving the salt into deionized water. Ammonium perrhenate (NH₄ReO₄) promoter solution is prepared by dissolving the salt in deionized water that is gently heated to 40-50° C. while stirring.

### Catalyst Synthesis by Vacuum Impregnation

The catalysts of examples 1-10 are synthesized by vacuum impregnation. The synthesis apparatus consists of a lower vacuum vessel that can be sealed on the top by a Teflon stopper connected to a second vessel with a stopcock. The synthesis starts by loading the bare alumina-containing carrier pellets into the lower vacuum vessel. The lower vessel is then sealed and placed under vacuum for 15 minutes. After the evacuation, the silver impregnation solution with the desired promoter concentrations is added to the top vessel. The stopcock is opened to introduce the promoted silver solution to the carrier under vacuum. The vacuum is then released, and the carrier is left immersed in the impregnation solution for 15 minutes, and subsequently drained for another 15 minutes. The newly impregnated carrier is placed in a single layer on a stainless-steel mesh tray and calcined at 500° C. for 10 minutes in an air oven. The catalyst is cooled and weighed to estimate the Ag loading after impregnation.

### Catalyst Synthesis by Incipient Wetness Impregnation

The catalysts of examples 11-45 are synthesized by an incipient wetness impregnation method. Unpromoted silver impregnated pills (prepared using a vacuum impregnation method similar to that described above using carrier A; 21.5 wt. % silver) are crushed and sieved to 30-50 mesh, divided into lots of 500 mg, and placed into synthesis tubes. Promoter solutions are prepared using deionized water, cesium hydroxide, lithium acetate, sodium acetate, ammonium sulfate, ammonium perrhenate, and manganese nitrate tetrahydrate. The manganese solution is stabilized with ethylenediaminetetraacetic acid diammonium salt and monoethanolamine. Promoter solutions are combined, and then added to the silver-impregnated powders, followed by mixing to achieve uniformity. After the impregnation, the samples are dried at 80° C. for 30 minutes, and then calcined at 500° C. for 10 minutes in a box oven under air flow.

### Elemental Analysis for Catalysts Synthesized by Vacuum Impregnation

For silver, cesium, sulfate, rhenium, and manganese, the elemental analyses were carried out by x-ray fluorescence spectrometry (XRF). For lithium and sodium, the elemental analyses were carried out by inductively coupled plasma optical emission spectrometry (ICP-OES).

### Testing Protocol, Continuous Stirred Tank Reactors

For catalyst performance testing in back-mixed Berty-type autoclave reactors (RotoBerty), 30 cm³ (˜20 g) of catalyst is loaded. The reactor is heated to 245° C. under nitrogen flow. The feed gases are introduced as soon as temperature reaches 220° C. The reaction conditions are 7.1 standard cubic feet per hour (scfh) total flow (201 standard liters per hour), gas hour space velocity (GHSV) ˜6800 h-1, 275 psig total pressure (1900 kPa gauge), and gas inlet concentrations (by volume) of 30% C₂H₄, 0.7% C₂H₆, 8% O₂, 1% CO₂, 4-5 ppm ethyl chloride (ECL), balance nitrogen. The catalyst is operated at these “break-in” conditions for 2-3 days unless otherwise specified in the following examples. After catalyst activation, the temperature was decreased to 235° C. and a gas-phase promoter optimization is carried out by a varying the inlet ethyl chloride concentration from low to high. The Cl optimization can also be conducted using outlet concentration control at 28.3% C₂H₄, 6.4% 02 and 1.5% CO₂. At each ethyl chloride concentration, the performance is allowed to stabilize, and an average value is noted. The optimum performance is reported as the selectivity and activity (ΔEO) at the inlet ECl concentration where selectivity is maximized. ΔEO is the difference between the outlet and inlet ethylene oxide concentrations, corrected for the change in molar volume across the reactor, measured in mole percent. It is calculated from the reactor inlet and outlet concentrations in mole percent of ethylene oxide (EOᵢₙₗₑₜ and EOₒᵤₜₗₑₜ, respectively) as follows: ΔEO %=SF×EOₒᵤₜₗₑₜ−EOᵢₙₗₑₜ. The term “SF” or “Shrink Factor” represents the net volumetric reduction occurring due to the production of the ethylene oxide. For every mole of ethylene oxide produced, there is a net reduction of 0.5 moles of total gas resulting in a corresponding reduction in the volumetric flow rate. The SF is typically calculated as follows: (200+EOᵢₙₗₑₜ)÷(200+EOₒᵤₜₗₑₜ), where EOᵢₙₗₑₜ and EOₒᵤₜₗₑₜ are the concentrations in mole percent of ethylene oxide in the reactor inlet and outlet gas mixtures, respectively.

### Testing Protocol, Plug Flow Reactors

The catalytic tests for high throughput evaluation were conducted in a High Pressure Reactor Assembly Module (HPRAM) system, described elsewhere, for example, U.S. Pat. No. 9,649,621. The HPRAM reactor system includes gas feed systems, 48 reactors, 2 exit modules, and 3 analyzers (Siemens MAXUM-II gas chromatographs (GCs)). Of the 48 reactors, seven are left blank to determine gas inlet concentrations.

The tests are carried out at constant catalyst bed volume (Vcatbed=0.1498 cm³), constant flow (19.6 standard cubic centimeters per minute), and constant gas hourly space velocity (GHSV=7850/hr.). Catalysts are charged into reactors in the form of a powder (30/50 mesh) without inert diluents. Catalysts are loaded into reactor tubes by mass, using the formula given below,

load mass (mg)=Vcatbed·PDcar·100%/(100%−AGWT)

where PDcar is the packing density of the carrier as listed in Table 1, and AGWT is the silver content of the catalyst in wt. %. Note that the use of whole pill packing densities for these tests is a model for tests at larger scale. The catalyst load masses are 100.0 mg and 93.8-100.3 mg for the tests reported in Table 6 and Table 8, respectively.

Reactors are charged with catalysts, and then heated under inert gas flow (either helium or nitrogen), and subsequently the feed gases are introduced to the reactors, except oxygen. Oxygen is added last (typically after 2-3 min), to avoid any chance of composing a flammable mixture in the system. The gas pressure and gas flow are then held constant at 10 barg and 19.6 standard cubic centimeters per minute for the duration of the tests.

The catalysts tests reported in Table 6 (catalysts 11-45) are carried out as follows. After an activation period of 2 days (245° C., 28 vol. % inlet ethylene, 4.8 vol. % inlet oxygen, 2.0 vol. % inlet carbon dioxide, 4 ppmv inlet ethyl chloride, 0.14 vol. % inlet ethane, 11 vol. % inlet methane, balance inert), the temperature is dropped to 235° C., and the gas inlets are adjusted to 32 vol. % ethylene, 7.6 vol. % oxygen, 1.5 vol. % carbon dioxide, 0.14 vol. % ethane, 11 vol. % methane, and varying amounts of the ethyl chloride promoter. The gas phase promoter optimization is carried out in plateau steps from low to high, with ethyl chloride ranging from 0.5 to 6.0 ppmv.

The catalysts tests reported in Table 8 (catalysts 46-57) are for averages over N=2-5 reactors. These tests are carried out as follows. After an activation period of 2 days (245° C., 27 vol. % inlet ethylene, 4.7 vol. % inlet oxygen, 1.7 vol. % inlet carbon dioxide, 1.4 ppmv inlet ethyl chloride, 0.12 vol. % inlet ethane, 11 vol. % inlet methane, balance inert), the gas inlets are adjusted to 32 vol. % ethylene, 7.4 vol. % oxygen, 1.3 vol. % carbon dioxide, 1.8 vol. % ethane, 0.7 ppmv ethyl chloride, and 11 vol. % methane. After 5 hours operation (T=245° C. and 1.8 vol. % ethane), the temperature is decreased to 235° C. and the ethane inlet is decreased to 0.12 vol. %. Then the gas phase promoter optimization is carried out in plateau steps from low to high, with ethyl chloride ranging from 0.96 to 3.84 ppmv.

### Catalysts Produced

### Catalyst 1

50 g of carrier A is converted into “Catalyst 1” according to the vacuum impregnation methods presented above. The following amounts are used in production of the impregnation solution:
200 g silver solution (27.41% Ag)
0.1987 g of Mn(NO₃)₂ solution (0.1570 g Mn/g solution)
1.0963 g (NH₄)₂H₂ (EDTA) solution (0.4030 g EDTA/g solution)
2.4931 g CsOH solution (0.1100 g Cs/g solution)
0.6179 g LiOCOCH₃ solution (0.0255 g Li/g solution)
0.3472 g NaOCOCH₃ solution (0.0551 g Na/g solution)
0.1238 g (NH₄)₂SO₄ solution (0.2908 g SO₄/g solution)
9.4962 g NH₄ReO₄ solution (0.0320 g Re/g solution)
   The catalyst produced comprises 19.2% Ag by weight, as measured by XRF. The target promoter concentrations for all promoters are listed in Table 3. Table 4 gives a comparison of the target promoter concentrations against the analyzed promoter concentrations.

### Catalyst 2

50 g of carrier B is converted into “Catalyst 2” according to the vacuum impregnation methods presented above. The following amounts are used in production of the impregnation solution:
200 g silver solution (27.41% Ag)
0.1912 g of Mn(NO₃)₂ solution (0.1570 g Mn/g solution)
1.0551 g (NH₄)₂H₂(EDTA) solution (0.4030 g EDTA/g solution)
2.9531 g CsOH solution (0.1100 g Cs/g solution)
0.7319 g LiOCOCH₃ solution (0.0255 g Li/g solution)
0.4551 g NaOCOCH₃ solution (0.0498 g Na/g solution)
0.1467 g (NH₄)₂SO₄ solution (0.2908 g SO₄/g solution)
11.1440 g NH₄ReO₄ solution (0.0323 g Re/g solution)
   The catalyst produced comprises 20% Ag by weight, as determined gravimetrically. The target promoter concentrations for all promoters are listed in Table 3.

### Catalyst 3

50 g of carrier C is converted into “Catalyst 3” according to the vacuum impregnation methods presented above. The following amounts are used in production of the impregnation solution:
200 g silver solution (26.8% Ag)
0.1883 g of Mn(NO₃)₂ solution (0.1560 g Mn/g solution)
1.0399 g (NH₄)₂H₂(EDTA) solution (0.4001 g EDTA/g solution)
1.5583 g CsOH solution (0.1090 g Cs/g solution)
0.6911 g LiOCOCH₃ solution (0.0255 g Li/g solution)
0.4214 g NaOCOCH₃ solution (0.0491 g Na/g solution)
0.0920 g (NH₄)₂SO₄ solution (0.2944 g SO₄/g solution)
5.2368 g NH₄ReO₄ solution (0.0318 g Re/g solution)
   The catalyst produced comprises 21% Ag by weight, as determined gravimetrically. The target promoter concentrations for all promoters are listed in Table 3.

### Catalyst 4

50 g of carrier C was converted into “Catalyst 4” according to the vacuum impregnation methods presented above. The following amounts are used in production of the impregnation solution:
200 g silver solution (26.8% Ag)
0.1882 g of Mn(NO₃)₂ solution (0.1560 g Mn/g solution)
1.0397 g (NH₄)₂H₂(EDTA) solution (0.4001 g EDTA/g solution)
1.8695 g CsOH solution (0.1090 g Cs/g solution)
1.7325 g LiOCOCH₃ solution (0.0255 g Li/g solution)
0.2407 g NaOCOCH₃ solution (0.0491 g Na/g solution)
0.1002 g (NH₄)₂SO₄ solution (0.2944 g SO₄/g solution)
5.2689 g NH₄ReO₄ solution (0.0316 g Re/g solution)
   The catalyst produced comprises 20% Ag by weight, as determined gravimetrically. The target promoter concentrations for all promoters are listed in Table 3.

### Catalyst 5

50 g of carrier C is converted into “Catalyst 5” according to the vacuum impregnation methods presented above. The following amounts are used in production of the impregnation solution:
200 g silver solution (27.3% Ag)
0.1921 g of Mn(NO₃)₂ solution (0.1560 g Mn/g solution)
1.0607 g (NH₄)₂H₂(EDTA) solution (0.4001 g EDTA/g solution)
1.9429 g CsOH solution (0.1105 g Cs/g solution)
1.0626 g LiOCOCH₃ solution (0.0255 g Li/g solution)
0.3661 g NaOCOCH₃ solution (0.0491 g Na/g solution)
0.1018 g (NH₄)₂SO₄ solution (0.2944 g SO₄/g solution)
6.9093 g NH₄ReO₄ solution (0.0316 g Re/g solution)
   The catalyst produced comprises of 19.7% Ag by weight, as measured by XRF. The target promoter concentrations for all promoters are listed in Table 3. Table 4 gives a comparison of the target promoter concentrations against the analyzed promoter concentrations.

### Catalyst 6

50 g of carrier D is converted into “Catalyst 6” according to the vacuum impregnation methods presented above. The following amounts are used in production of the impregnation solution:
200 g silver solution (27.37% Ag)
0.1926 g of Mn(NO₃)₂ solution (0.1560 g Mn/g solution)
1.0639 g (NH₄)₂H₂(EDTA) solution (0.4001 g EDTA/g solution)
1.9487 g CsOH solution (0.1105 g Cs/g solution)
1.3732 g LiOCOCH₃ solution (0.0255 g Li/g solution)
0.5383 g NaOCOCH₃ solution (0.0500 g Na/g solution)
0.1429 g (NH₄)₂SO₄ solution (0.2944 g SO₄/g solution)
7.8562 g NH₄ReO₄ solution (0.0316 g Re/g solution)
   The catalyst produced comprises of 19.5% Ag by weight, as measured by XRF. The target promoter concentrations for all promoters are listed in Table 3. Table 4 gives a comparison of the target promoter concentrations against the analyzed promoter concentrations.

### Catalyst 7

50 g of carrier F is converted into “Catalyst 7” according to the vacuum impregnation methods presented above. The following amounts are used in production of the impregnation solution:
200 g silver solution (27.37% Ag)
0.2020 g of Mn(NO₃)₂ solution (0.1560 g Mn/g solution)
1.1154 g (NH₄)₂H₂(EDTA) solution (0.4001 g EDTA/g solution)
1.8693 g CsOH solution (0.1105 g Cs/g solution)
1.0528 g LiOCOCH₃ solution (0.0255 g Li/g solution)
0.2411 g NaOCOCH₃ solution (0.0500 g Na/g solution)
0.1014 g (NH₄)₂SO₄ solution (0.2944 g SO₄/g solution)
5.3403 g NH₄ReO₄ solution (0.0316 g Re/g solution)
   The catalyst produced comprises of 16.5% Ag by weight, as measured by XRF. The target promoter concentrations for all promoters are listed in Table 3. Table 4 gives a comparison of the target promoter concentrations against the analyzed promoter concentrations.

### Catalyst 8

50 g of carrier E is converted into “Catalyst 8” according to the vacuum impregnation methods presented above. The following amounts are used in production of the impregnation solution:
200 g silver solution (27.3% Ag)
0.2017 g of Mn(NO₃)₂ solution (0.1560 g Mn/g solution)
1.1140 g (NH₄)₂H₂(EDTA) solution (0.4001 g EDTA/g solution)
1.9760 g CsOH solution (0.1105 g Cs/g solution)
1.1105 g LiOCOCH₃ solution (0.0255 g Li/g solution)
0.2579 g NaOCOCH₃ solution (0.0491 g Na/g solution)
0.1074 g (NH₄)₂SO₄ solution (0.2944 g SO₄/g solution)
5.6454 g NH₄ReO₄ solution (0.0316 g Re/g solution)
   The catalyst produced comprises of 17.1% Ag by weight, as measured by XRF. The target promoter concentrations for all promoters are listed in Table 3. Table 4 gives a comparison of the target promoter concentrations against the analyzed promoter concentrations.

### Catalyst 9

50 g of carrier D is converted into “Catalyst 9” according to the vacuum impregnation methods presented above, except for a change in the calcination treatment. Following impregnation and draining, the wet pills are treated in an air oven at 110° C. for 10 minutes prior to calcination at 500° C. for 10 minutes. The following amounts are used in production of the impregnation solution:
200 g silver solution (27.37% Ag)
0.2022 g of Mn(NO₃)₂ solution (0.1560 g Mn/g solution)
1.1167 g (NH₄)₂H₂(EDTA) solution (0.4001 g EDTA/g solution)
1.6208 g CsOH solution (0.1105 g Cs/g solution)
0.7314 g LiOCOCH₃ solution (0.0255 g Li/g solution)
0.4388 g NaOCOCH₃ solution (0.0500 g Na/g solution)
0.0969 g (NH₄)₂SO₄ solution (0.2944 g SO₄/g solution)
5.5549 g NH₄ReO₄ solution (0.0316 g Re/g solution)
   The catalyst produced comprises of 21.1% Ag by weight, as measured by XRF. The target promoter concentrations for all promoters are listed in Table 3. Table 4 gives a comparison of the target promoter concentrations against the analyzed promoter concentrations.

### Catalyst 10

50 g of carrier D is converted into “Catalyst 10” according to the vacuum impregnation methods presented above, except for a change in the calcination treatment. Following impregnation and draining, the wet pills are treated in an air oven at 90° C. for 90 minutes prior to calcination at 500° C. for 10 minutes. The following amounts are used in production of the impregnation solution:
200 g silver solution (27.37% Ag)
0.1901 g of Mn(NO₃)₂ solution (0.1560 g Mn/g solution)
1.0502 g (NH₄)₂H₂(EDTA) solution (0.4001 g EDTA/g solution)
1.5243 g CsOH solution (0.1105 g Cs/g solution)
0.6878 g LiOCOCH₃ solution (0.0255 g Li/g solution)
0.4127 g NaOCOCH₃ solution (0.0500 g Na/g solution)
0.0911 g (NH₄)₂SO₄ solution (0.2944 g SO₄/g solution)
5.2241 g NH₄ReO₄ solution (0.0316 g Re/g solution)
   The catalyst produced comprises of 22.4% Ag by weight, as measured by XRF. The target promoter concentrations for all promoters are listed in Table 2. Table 3 gives a comparison of the target promoter concentrations against the analyzed promoter concentrations.

TABLE 2
Target synthesis promoter concentrations for catalysts 1-10

| | | Cs | Li | Na | SO4 | Re | | F1 | F2 |
|---|---|---|---|---|---|---|---|---|---|
| | | (mmol/ | (mmol/ | (mmol/ | (mmol/ | (mmol/ | Mn | (mmol/ | (mmol/ |
| catalyst | carrier | kg) | kg) | kg) | kg) | kg) | (ppm) | kg) | kg) |
| 1 | A | 7.61 | 8.36 | 3.09 | 1.38 | 6.01 | 115 | 2.68 | 5.18 |
| 2 | B | 9.36 | 10.37 | 3.78 | 1.70 | 7.41 | 115 | 3.29 | 6.36 |
| 3 | C | 5.00 | 9.94 | 3.52 | 1.10 | 3.50 | 115 | 2.89 | 2.00 |
| 4 | C | 6.00 | 24.92 | 2.00 | 1.21 | 3.50 | 115 | 3.58 | −0.16 |
| 5 | C | 6.20 | 14.98 | 3.00 | 1.20 | 4.50 | 115 | 3.00 | 2.15 |
| 6 | D | 6.20 | 19.31 | 4.48 | 1.68 | 5.10 | 115 | 2.92 | 0.86 |
| 7 | F | 5.67 | 14.12 | 1.91 | 1.13 | 3.31 | 115 | 3.10 | 2.11 |
| 8 | E | 6.00 | 14.98 | 2.00 | 1.21 | 3.50 | 115 | 3.26 | 2.23 |
| 9 | D | 4.91 | 9.80 | 3.48 | 1.08 | 3.44 | 115 | 2.85 | 1.95 |
| 10 | D | 4.91 | 9.80 | 3.48 | 1.08 | 3.44 | 115 | 2.85 | 1.95 |

TABLE 3
Promoter concentrations, analyses vs. synthesis targets

| cata- | | Cs | Li | Na | SO₄ | Re | Mn |
|---|---|---|---|---|---|---|---|
| lyst | method | (ppm) | (ppm) | (ppm) | (ppm) | (ppm) | (ppm) |
| 1 | analysis | 1008 | 60 | 79 | 132 | 1117 | 117 |
| 5 | analysis | 788 | 109 | 95 | 145 | 843 | 116 |
| 6 | analysis | 837 | 136 | 112 | 156 | 922 | 115 |
| 7 | analysis | 700 | 90 | 54 | 109 | 536 | 92 |
| 8 | analysis | 743 | 99 | 53 | 109 | 588 | 106 |
| 9 | analysis | 733 | 76 | 95 | 108 | 676 | 128 |
| 10 | analysis | 724 | 72 | 93 | 110 | 686 | 125 |
| 1 | target | 1011 | 58 | 71 | 133 | 1120 | 115 |
| 5 | target | 824 | 104 | 69 | 115 | 838 | 115 |
| 6 | target | 824 | 134 | 103 | 161 | 950 | 115 |
| 7 | target | 754 | 98 | 44 | 109 | 616 | 115 |
| 8 | target | 798 | 104 | 46 | 116 | 652 | 115 |
| 9 | target | 653 | 68 | 80 | 104 | 640 | 115 |
| 10 | target | 653 | 68 | 80 | 104 | 640 | 115 |
| 1 | delta | −3 | 2 | 8 | −1 | −3 | 2 |
| 5 | delta | −36 | 5 | 26 | 30 | 5 | 1 |
| 6 | delta | 13 | 2 | 9 | −5 | −28 | 0 |
| 7 | delta | −54 | −8 | 10 | 0 | −80 | −23 |
| 8 | delta | −55 | −5 | 7 | −7 | −64 | −9 |
| 9 | delta | 80 | 8 | 15 | 4 | 36 | 13 |
| 10 | delta | 71 | 4 | 13 | 6 | 46 | 10 |

Table 4 shows the catalytic performance using the CSTR test protocol given above for catalysts 1-10. All 10 catalysts are inventive, and each achieves a selectivity no less than 88.4% and an activity no less than ΔEO=1.23 vol. %.

TABLE 4
Catalyst performance, catalysts 1-10.

| | Selectivity, | ΔEO, |
|---|---|---|
| Catalyst | % | vol. % |
| 1 | 88.8 | 1.23 |
| 2 | 88.4 | 1.36 |
| 3 | 89.1 | 1.48 |
| 4 | 88.9 | 1.45 |
| 5 | 88.9 | 1.5 |
| 6 | 88.6 | 1.44 |
| 7 | 88.5 | 1.5 |
| 8 | 89.9 | 1.41 |
| 9 | 89.2 | 1.45 |
| 10 | 88.7 | 1.54 |

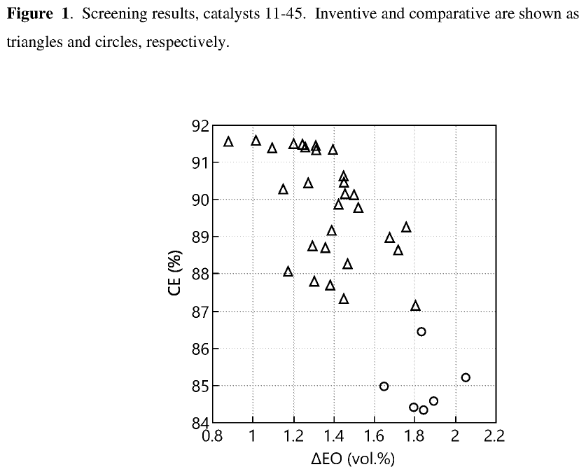

Catalysts 11-45 were prepared using the incipient wetness method presented above, and the performance of catalysts 11-45 were evaluated in the HPRAM reactor following the testing protocol for plug-flow reactors. The target promoter concentrations are given in Table 5. The performance results are given in Table 6 and in FIG. 1. As shown in FIG. 1, the selectivities are in the range of 87.1-91.6% and 84.3-86.4% for the inventive and comparative catalysts, respectively.

TABLE 5
Target promoter concentrations of catalysts 11-45

| | | Cs/Q | Li/Q | Na/Q | S/Q | Re/Q | | F1/Q | F2/Q |
|---|---|---|---|---|---|---|---|---|---|
| | | (mmol/ | (mmol/ | (mmol/ | (mmol/ | (mmol/ | Mn | (mmol/ | (mmol/ |
| catalyst | type | kg) | kg) | kg) | kg) | kg) | (ppm) | kg) | kg) |
| 11 | inv | 5.34 | 19.40 | 2.59 | 1.45 | 3.88 | 115 | 2.49 | 0.43 |
| 12 | inv | 5.34 | 12.93 | 2.59 | 1.45 | 3.10 | 115 | 3.01 | 1.98 |
| 13 | inv | 5.34 | 12.93 | 2.59 | 1.03 | 3.88 | 115 | 2.58 | 1.85 |
| 14 | inv | 5.34 | 12.93 | 1.29 | 1.45 | 3.88 | 115 | 1.68 | 2.33 |
| 15 | inv | 5.34 | 12.93 | 2.59 | 0.62 | 4.66 | 115 | 2.15 | 1.73 |
| 16 | inv | 5.34 | 12.93 | 3.88 | 1.45 | 3.88 | 115 | 2.89 | 1.63 |
| 17 | inv | 5.34 | 6.47 | 2.59 | 0.62 | 3.88 | 115 | 2.67 | 3.28 |
| 18 | inv | 5.80 | 8.88 | 3.05 | 1.47 | 4.59 | 149 | 2.15 | 3.29 |
| 19 | inv | 4.29 | 8.88 | 3.05 | 1.47 | 4.59 | 81 | 0.64 | 1.77 |
| 20 | inv | 5.80 | 8.88 | 1.02 | 1.47 | 3.39 | 81 | 2.32 | 3.84 |
| 21 | inv | 6.31 | 8.33 | 1.02 | 1.37 | 2.99 | 58 | 3.25 | 4.45 |
| 22 | inv | 6.31 | 2.78 | 3.05 | 1.37 | 2.99 | 173 | 4.03 | 5.23 |
| 23 | inv | 6.31 | 8.33 | 3.05 | 1.37 | 4.99 | 58 | 2.33 | 3.90 |
| 24 | inv | 4.31 | 14.22 | 3.02 | 0.91 | 3.02 | 115 | 2.69 | 0.36 |
| 25 | comp | 5.39 | 12.93 | 4.53 | 0.46 | 2.26 | 115 | 5.47 | 1.20 |
| 26 | inv | 4.31 | 8.62 | 1.06 | 0.91 | 3.02 | 115 | 1.59 | 2.23 |
| 27 | comp | 3.23 | 12.93 | 4.53 | 0.46 | 2.26 | 115 | 3.32 | −0.96 |
| 28 | inv | 6.47 | 30.17 | 0.86 | 1.03 | 3.77 | 115 | 3.55 | −0.70 |
| 29 | inv | 5.17 | 21.55 | 0.60 | 1.03 | 3.02 | 115 | 2.56 | 0.15 |
| 30 | comp | 3.88 | 30.17 | 2.59 | 1.03 | 2.26 | 115 | 3.19 | −3.75 |
| 31 | comp | 6.47 | 12.93 | 2.59 | 1.03 | 2.26 | 115 | 5.22 | 2.97 |
| 32 | comp | 6.47 | 30.17 | 0.86 | 1.03 | 2.26 | 115 | 4.96 | −0.70 |
| 33 | inv | 5.44 | 23.29 | 5.43 | 0.88 | 3.30 | 115 | 5.01 | −1.35 |
| 34 | inv | 6.68 | 16.64 | 5.43 | 2.05 | 5.50 | 115 | 3.12 | 1.84 |
| 35 | comp | 5.26 | 21.55 | 3.19 | 2.76 | 5.66 | 115 | 0.15 | 0.05 |
| 36 | inv | 5.26 | 21.55 | 4.92 | 1.90 | 4.31 | 115 | 2.84 | −0.67 |
| 37 | inv | 6.29 | 21.55 | 4.91 | 2.76 | 5.18 | 115 | 2.44 | 0.62 |
| 38 | inv | 5.78 | 21.55 | 4.91 | 1.90 | 5.17 | 115 | 2.55 | −0.15 |
| 39 | inv | 5.77 | 21.55 | 6.64 | 1.89 | 5.17 | 115 | 3.36 | −0.63 |
| 40 | inv | 5.26 | 21.55 | 6.64 | 1.03 | 6.03 | 115 | 2.65 | −1.40 |
| 41 | inv | 5.74 | 21.55 | 6.64 | 2.76 | 5.17 | 115 | 2.70 | −0.40 |
| 42 | inv | 6.29 | 21.55 | 6.64 | 2.76 | 4.31 | 115 | 4.06 | 0.16 |
| 43 | inv | 6.29 | 21.55 | 3.19 | 2.76 | 4.31 | 115 | 2.44 | 1.09 |
| 44 | inv | 5.26 | 21.55 | 6.64 | 2.76 | 4.31 | 115 | 3.03 | −0.88 |
| 45 | inv | 6.29 | 21.55 | 3.19 | 1.90 | 5.19 | 115 | 2.24 | 0.83 |

TABLE 6
Catalytic performance of catalysts 11-45

| | | selectivity, | ΔEO, |
|---|---|---|---|
| catalyst | type | % | vol. % |
| 11 | inv | 89.2 | 1.76 |
| 12 | inv | 89.0 | 1.68 |
| 13 | inv | 90.1 | 1.50 |
| 14 | inv | 89.8 | 1.52 |
| 15 | inv | 90.1 | 1.46 |
| 16 | inv | 90.4 | 1.45 |
| 17 | inv | 89.9 | 1.42 |
| 18 | inv | 88.7 | 1.30 |
| 19 | inv | 87.3 | 1.45 |
| 20 | inv | 88.3 | 1.47 |
| 21 | inv | 89.1 | 1.39 |
| 22 | inv | 88.0 | 1.18 |
| 23 | inv | 90.3 | 1.15 |
| 24 | inv | 88.6 | 1.72 |
| 25 | comp | 84.4 | 1.80 |
| 26 | inv | 87.7 | 1.38 |
| 27 | comp | 86.4 | 1.83 |
| 28 | inv | 88.7 | 1.36 |
| 29 | inv | 87.1 | 1.80 |
| 30 | comp | 85.2 | 2.05 |
| 31 | comp | 84.3 | 1.84 |
| 32 | comp | 84.6 | 1.89 |
| 33 | inv | 87.8 | 1.30 |
| 34 | inv | 91.5 | 1.20 |
| 35 | comp | 85.0 | 1.65 |
| 36 | inv | 91.4 | 1.26 |
| 37 | inv | 91.3 | 1.31 |
| 38 | inv | 91.3 | 1.40 |
| 39 | inv | 91.4 | 1.31 |
| 40 | inv | 90.6 | 1.45 |
| 41 | inv | 91.5 | 1.25 |
| 42 | inv | 91.5 | 0.88 |
| 43 | inv | 91.4 | 1.10 |
| 44 | inv | 91.6 | 1.02 |
| 45 | inv | 90.4 | 1.27 |

TABLE 7
Target promoter concentrations of catalysts 46-57

| | | Cs | Li | Na | SO4 | Re | Mn | | |
|---|---|---|---|---|---|---|---|---|---|
| example | type | (ppm) | (ppm) | (ppm) | (ppm) | (ppm) | (ppm) | F1/Q | F2/Q |
| 46 | inv | 801 | 130 | 100 | 157 | 924 | 112 | 2.4 | 0.7 |
| 47 | inv | 836 | 136 | 105 | 163 | 964 | 0 | 2.6 | 0.8 |
| 48 | inv | 794 | 129 | 99 | 155 | 915 | 265 | 2.4 | 0.7 |
| 49 | inv | 860 | 140 | 108 | 168 | 991 | 203 | 2.6 | 0.8 |
| 50 | inv | 857 | 139 | 107 | 167 | 988 | 59 | 2.6 | 0.8 |
| 51 | inv | 832 | 0 | 104 | 162 | 959 | 116 | 2.0 | 4.8 |
| 52 | inv | 880 | 0 | 110 | 172 | 1014 | 0 | 2.1 | 5.1 |
| 53 | inv | 809 | 0 | 158 | 207 | 933 | 113 | 2.6 | 4.2 |
| 54 | comp | 486 | 27 | 34 | 117 | 341 | 73 | 1.5 | 2.1 |
| 55 | comp | 495 | 28 | 35 | 119 | 347 | 74 | 1.5 | 2.1 |
| 56 | inv | 858 | 140 | 108 | 168 | 989 | 109 | 2.4 | 0.7 |
| 57 | inv | 908 | 148 | 114 | 178 | 1047 | 115 | 2.5 | 0.7 |

TABLE 8
Catalytic performance of catalysts 46-57

| | | | selectivity | ΔEO |
|---|---|---|---|---|
| catalyst | type | N | (%) | (vol. %) |
| 46 | inv | 5 | 90.5 | 1.30 |
| 47 | inv | 3 | 91.5 | 1.23 |
| 48 | inv | 3 | 90.1 | 1.16 |
| 49 | inv | 3 | 90.3 | 1.19 |
| 50 | inv | 3 | 90.9 | 1.16 |
| 51 | inv | 3 | 86.7 | 0.99 |
| 52 | inv | 3 | 87.1 | 0.98 |
| 53 | inv | 3 | 88.6 | 1.22 |
| 54 | comp | 2 | 84.9 | 1.02 |
| 55 | comp | 2 | 86.3 | 1.23 |
| 56 | inv | 3 | 89.4 | 1.16 |
| 57 | inv | 3 | 91.0 | 1.16 |

### Catalyst 46

50.09 g of carrier G is converted into “Catalyst 46” according to the vacuum impregnation methods presented above. The following amounts are used in production of the impregnation solution:
165.04 g silver solution (25.69 wt. % Ag)
0.6013 g of Mn(NO3)2 solution (38.00 mg Mn/g solution)
0.8065 g (NH4)2H2 (EDTA) solution (401.0 mg EDTA/g solution)
1.4623 g CsOH solution (111.9 mg Cs/g solution)
2.2177 g LiOCOCH3 solution (12.00 mg Li/g solution)
0.4091 g NaOCOCH3 solution (50.00 mg Na/g solution)
0.7992 g (NH4)2SO4 solution (40.00 mg SO4/g solution)
5.8594 g NH4ReO4 solution (32.20 g Re/g solution). The catalyst produced comprises 21.02 wt. % Ag, as determined gravimetrically. The target promoter concentrations for all promoters are listed in Table 7.

### Catalyst 47

50.21 g of carrier G is converted into “Catalyst 47” according to the vacuum impregnation methods presented above. The following amounts are used in production of the impregnation solution:
165.35 g silver solution (27.62 wt. % Ag)
0.0000 g of Mn(NO3)2 solution (38.00 mg Mn/g solution)
0.0000 g (NH4)2H2 (EDTA) solution (401.0 mg EDTA/g solution)
1.6822 g CsOH solution (111.9 mg Cs/g solution)
2.5511 g LiOCOCH3 solution (12.00 mg Li/g solution)
0.4704 g NaOCOCH3 solution (50.00 mg Na/g solution)
0.9195 g (NH4)2SO4 solution (40.00 mg SO4/g solution)
6.7391 g NH4ReO4 solution (32.20 g Re/g solution). The catalyst produced comprises 20.54 wt. % Ag, as determined gravimetrically. The target promoter concentrations for all promoters are listed in Table 7.

### Catalyst 48

50.08 g of carrier G is converted into “Catalyst 48” according to the vacuum impregnation methods presented above. The following amounts are used in production of the impregnation solution:
165.20 g silver solution (25.69 wt. % Ag)
1.5226 g of Mn(NO3)2 solution (38.00 mg Mn/g solution)
2.0444 g (NH4)2H2 (EDTA) solution (401.0 mg EDTA/g solution)
1.5497 g CsOH solution (111.9 mg Cs/g solution)
2.3497 g LiOCOCH3 solution (12.00 mg Li/g solution)
0.4337 g NaOCOCH3 solution (50.00 mg Na/g solution)
0.8471 g (NH4)2SO4 solution (40.00 mg SO4/g solution)
6.2084 g NH4ReO4 solution (32.20 g Re/g solution). The catalyst produced comprises 19.69 wt. % Ag, as determined gravimetrically. The target promoter concentrations for all promoters are listed in Table 7.

### Catalyst 49

50.16 g of carrier G is converted into “Catalyst 49” according to the vacuum impregnation methods presented above. The following amounts are used in production of the impregnation solution:
165.25 g silver solution (25.69 wt. % Ag)
1.0960 g of Mn(NO3)2 solution (38.00 mg Mn/g solution)
1.4711 g (NH4)2H2 (EDTA) solution (401.0 mg EDTA/g solution)
1.5730 g CsOH solution (111.9 mg Cs/g solution)
2.3847 g LiOCOCH3 solution (12.00 mg Li/g solution)
0.4403 g NaOCOCH3 solution (50.00 mg Na/g solution)
0.8597 g (NH4)2SO4 solution (40.00 mg SO4/g solution)
6.3016 g NH4ReO4 solution (32.20 g Re/g solution). The catalyst produced comprises 21.02 wt. % Ag, as determined gravimetrically. The target promoter concentrations for all promoters are listed in Table 7.

### Catalyst 50

50.23 g of carrier G is converted into “Catalyst 50” according to the vacuum impregnation methods presented above. The following amounts are used in production of the impregnation solution:
165.01 g silver solution (25.69 wt. % Ag)
0.3035 g of Mn(NO3)2 solution (38.00 mg Mn/g solution)
0.4073 g (NH4)2H2 (EDTA) solution (401.0 mg EDTA/g solution)
1.4903 g CsOH solution (111.9 mg Cs/g solution)
2.2595 g LiOCOCH3 solution (12.00 mg Li/g solution)
0.4168 g NaOCOCH3 solution (50.00 mg Na/g solution)
0.8144 g (NH4)2SO4 solution (40.00 mg SO4/g solution)
5.9709 g NH4ReO4 solution (32.20 g Re/g solution). The catalyst produced comprises 22.06 wt. % Ag, as determined gravimetrically. The target promoter concentrations for all promoters are listed in Table 7.

### Catalyst 51

50.10 g of carrier G is converted into “Catalyst 51” according to the vacuum impregnation methods presented above. The following amounts are used in production of the impregnation solution:
165.16 g silver solution (25.69 wt. % Ag)
0.6126 g of Mn(NO3)2 solution (38.00 mg Mn/g solution)
0.8222 g (NH4)2H2 (EDTA) solution (401.0 mg EDTA/g solution)
1.4901 g CsOH solution (111.9 mg Cs/g solution)
0.0000 g LiOCOCH3 solution (12.00 mg Li/g solution)
0.4169 g NaOCOCH3 solution (50.00 mg Na/g solution)
0.8144 g (NH4)2SO4 solution (40.00 mg SO4/g solution)
5.9702 g NH4ReO4 solution (32.20 g Re/g solution). The catalyst produced comprises 21.41 wt. % Ag, as determined gravimetrically. The target promoter concentrations for all promoters are listed in Table 7.

### Catalyst 52

50.41 g of carrier G is converted into “Catalyst 52” according to the vacuum impregnation methods presented above. The following amounts are used in production of the impregnation solution:
165.00 g silver solution (25.69 wt. % Ag)
0.0000 g of Mn(NO3)2 solution (38.00 mg Mn/g solution)
0.0000 g (NH4)2H2 (EDTA) solution (401.0 mg EDTA/g solution)
1.5414 g CsOH solution (111.9 mg Cs/g solution)
0.0000 g LiOCOCH3 solution (12.00 mg Li/g solution)
0.4314 g NaOCOCH3 solution (50.00 mg Na/g solution)
0.8422 g (NH4)2SO4 solution (40.00 mg SO4/g solution)
6.1754 g NH4ReO4 solution (32.20 g Re/g solution). The catalyst produced comprises 21.88 wt. % Ag, as determined gravimetrically. The target promoter concentrations for all promoters are listed in Table 7.

### Catalyst 53

50.35 g of carrier G is converted into “Catalyst 53” according to the vacuum impregnation methods presented above. The following amounts are used in production of the impregnation solution:
165.18 g silver solution (25.69 wt. % Ag)
0.6335 g of Mn(NO3)2 solution (38.00 mg Mn/g solution)
0.8507 g (NH4)2H2 (EDTA) solution (401.0 mg EDTA/g solution)
1.5416 g CsOH solution (111.9 mg Cs/g solution)
0.0000 g LiOCOCH3 solution (12.00 mg Li/g solution)
0.6742 g NaOCOCH3 solution (50.00 mg Na/g solution)
1.1044 g (NH4)2SO4 solution (40.00 mg SO4/g solution)
6.1766 g NH4ReO4 solution (32.20 g Re/g solution). The catalyst produced comprises 20.16 wt. % Ag, as determined gravimetrically. The target promoter concentrations for all promoters are listed in Table 7.

### Catalyst 54

50.34 g of carrier H is converted into “Catalyst 54” according to the vacuum impregnation methods presented above. For the preparation of this catalyst, the silver impregnation solution was prepared by diluting the silver impregnation solution that was used for Catalyst 50 with deionized water. The following amounts are used in production of the impregnation solution:
165.10 g silver solution (18.00 wt. % Ag)
0.3558 g of Mn(NO3)2 solution (38.00 mg Mn/g solution)
0.4771 g (NH4)2H2 (EDTA) solution (401.0 mg EDTA/g solution)
0.8036 g CsOH solution (111.9 mg Cs/g solution)
0.4207 g LiOCOCH3 solution (12.00 mg Li/g solution)
0.1264 g NaOCOCH3 solution (50.00 mg Na/g solution)
0.5409 g (NH4)2SO4 solution (40.00 mg SO4/g solution)
1.9589 g NH4ReO4 solution (32.20 g Re/g solution). The catalyst produced comprises 16.18 wt. % Ag, as determined gravimetrically. The target promoter concentrations for all promoters are listed in Table 7.

### Catalyst 55

50.13 g of carrier H is converted into “Catalyst 55” according to the vacuum impregnation methods presented above. For the preparation of this catalyst, the silver impregnation solution was prepared by diluting the silver impregnation solution that was used for Catalyst 50 with deionized water. The following amounts are used in production of the impregnation solution:
165.10 g silver solution (20.18 wt. % Ag)
0.3576 g of Mn(NO3)2 solution (38.00 mg Mn/g solution)
0.4798 g (NH4)2H2 (EDTA) solution (401.0 mg EDTA/g solution)
0.8080 g CsOH solution (111.9 mg Cs/g solution)
0.4228 g LiOCOCH3 solution (12.00 mg Li/g solution)
0.1263 g NaOCOCH3 solution (50.00 mg Na/g solution)
0.5438 g (NH4)2SO4 solution (40.00 mg SO4/g solution)
1.9690 g NH4ReO4 solution (32.20 g Re/g solution). The catalyst produced comprises 18.36 wt. % Ag, as determined gravimetrically. The target promoter concentrations for all promoters are listed in Table 7.

### Catalyst 56

50.35 g of carrier H is converted into “Catalyst 56” according to the vacuum impregnation methods presented above. For the preparation of this catalyst, the silver impregnation solution was prepared by diluting the silver impregnation solution that was used for Catalyst 50 with deionized water. The following amounts are used in production of the impregnation solution:
164.90 g silver solution (18.00 wt. % Ag)
0.5464 g of Mn(NO3)2 solution (38.00 mg Mn/g solution)
0.7333 g (NH4)2H2 (EDTA) solution (401.0 mg EDTA/g solution)
1.4664 g CsOH solution (111.9 mg Cs/g solution)
2.2263 g LiOCOCH3 solution (12.00 mg Li/g solution)
0.4117 g NaOCOCH3 solution (50.00 mg Na/g solution)
0.8033 g (NH4)2SO4 solution (40.00 mg SO4/g solution)
5.8757 g NH4ReO4 solution (32.20 g Re/g solution). The catalyst produced comprises 15.79 wt. % (that is, 16 wt. % when rounded to the nearest whole number) Ag, as determined gravimetrically. The target promoter concentrations for all promoters are listed in Table 7.

### Catalyst 57

50.22 g of carrier H is converted into “Catalyst 57” according to the vacuum impregnation methods presented above. For the preparation of this catalyst, the silver impregnation solution was prepared by diluting the silver impregnation solution that was used for Catalyst 50 with deionized water. The following amounts are used in production of the impregnation solution:
165.05 g silver solution (20.18 wt. % Ag)
0.5712 g of Mn(NO3)2 solution (38.00 mg Mn/g solution)
0.7661 g (NH4)2H2 (EDTA) solution (401.0 mg EDTA/g solution)
1.5326 g CsOH solution (111.9 mg Cs/g solution)
2.3264 g LiOCOCH3 solution (12.00 mg Li/g solution)
0.4297 g NaOCOCH3 solution (50.00 mg Na/g solution)
0.8394 g (NH4)2SO4 solution (40.00 mg SO4/g solution)
6.1408 g NH4ReO4 solution (32.20 g Re/g solution). The catalyst produced comprises 17.92 wt. % Ag, as determined gravimetrically. The target promoter concentrations for all promoters are listed in Table 7.

1. A supported silver catalyst prepared on an alumina-containing carrier, the carrier comprising greater than about 80 weight percent alpha-alumina and less than about 30 parts per million acid-leachable alkali metals by weight, the weight percent of the alumina and the concentration of the acid-leachable alkali metals being calculated on the weight of the carrier, wherein the acid-leachable alkali metals are selected from lithium, sodium, potassium, and mixtures thereof, the carrier having deposited thereon:
(A) silver in an amount of from 16 to 25 percent by weight of the catalyst; and
(B) a solid promoter package comprising cesium, sodium, sulfate, rhenium, and optionally lithium, wherein for these promoters, the amounts are expressed in units of millimoles of promoter per kilogram of catalyst, and the amounts of these promoters in the solid promoter package deposited on the catalyst are such that:
C_{Cs}/Q is in a range of from 3.1 to 8.7 mmol/kg catalyst;
C_{Na}/Q is in a range of from 0.5 to 7.5 mmol/kg catalyst;
C_{S}/Q is in a range of from 0.3 to 3.2 mmol/kg catalyst;
C_{Re}/Q is in a range of from 2.4 to 6.9 mmol/kg catalyst; and
C_{Li}/Q is in a range of from 0 to 35 mmol/kg catalyst; and
F1/Q is in a range of from 0.3 to 5.2 mmol/kg catalyst; and
F2/Q is in a range of from −5.1 to 6.3 mmol/kg catalyst;
where Q is a unitless scaling factor equal to the surface area of the alumina-containing carrier prior to deposition of silver and promoters expressed in units of square meters per gram divided by one square meter per gram;
where F1 and F2 are defined by the following equations:
$$F 1 = C {c}_{s} + 0.032 \cdot {C}_{L i} + 0.47 \cdot {C}_{N a} - ( 0.72 \cdot Cs + 0.94 \cdot {C}_{R e} ) ;$$
$$F 2 = C {c}_{s} - 0.24 \cdot {C}_{L i} - 0.27 \cdot {C}_{N a} + 0.3 \cdot Cs ; and$$
where C_{Cs}, C_{Li}, C_{Na}, C_{S}, and C_{Re} are the amounts of cesium, lithium, sodium, sulfate, and rhenium, respectively, deposited on the carrier expressed in units of mmol promoter per kg of catalyst.

2. The catalyst of claim 1 wherein the carrier further has deposited thereon manganese in an amount of from 20 to 300 ppm by weight of the catalyst.

3. The catalyst of claim 1 wherein the amount of cesium deposited on the catalyst is such that C_{Cs}/Q is in a range of from 4.2 to 7.8 mmol/kg catalyst.

4. The catalyst of claim 1, wherein the amount of lithium deposited on the catalyst is such that C_{Li}/Q is in a range of from 6 to 30 mmol/kg catalyst.

5. The catalyst of claim 1, wherein the amount of sodium deposited on the catalyst is such that C_{Na}/Q is in a range of from 1.2 to 7.5 mmol/kg catalyst.

6. The catalyst of claim 1, wherein the amount of rhenium deposited on the catalyst is such that C_{Re}/Q is in a range of from 3.0 to 6.8 mmol/kg catalyst.

7. The catalyst of claim 1, wherein the amounts of promoters deposited on the catalyst are such that F1/Q is in a range of from 1.5 to 4.1 mmol/kg catalyst.

8. The catalyst of claim 7 wherein the amounts of promoters deposited on the catalyst are such that F1/Q is in a range of from 1.9 to 3.7 mmol/kg catalyst.

9. The catalyst of claim 1, wherein the amounts of promoters deposited on the catalyst are such that F2/Q is in a range of from −2.5 to 3.5 mmol/kg catalyst.

10. The catalyst of claim 9 wherein the amounts of promoters deposited on the catalyst are such that F2/Q is in a range of from −1.8 to 2.7 mmol/kg catalyst.

11. The catalyst of claim 1 wherein the amounts of promoters in the solid promoter package deposited on the catalyst are such that:
C_{Cs}/Q is in a range of from 4.7 to 7.3 mmol/kg catalyst; and
C_{Li}/Q is in a range of from 10 to 26 mmol/kg catalyst; and
C_{Na}/Q is in a range of from 2.5 to 7.5 mmol/kg catalyst; and
C_{Re}/Q is in a range of from 3.3 to 6.7 mmol/kg catalyst; and wherein F1/Q is in a range of from 1.9 to 3.7 mmol/kg catalyst; and F2/Q is in a range of from −1.8 to 2.7 mmol/kg catalyst.

12. The catalyst of claim 1, wherein the catalyst is prepared using a calcination step which is carried out at a temperature within a range of 480-550° C.

13. The catalyst of claim 12 wherein the calcination is carried out on a roasting belt, wherein the catalyst is in a hot zone at a temperature within a range of 480-550° C. for a duration not greater than 5 minutes.

14. The catalyst of claim 1, wherein the alumina-containing carrier has a surface area in the range of 0.7 to 1.5 m²/g.

15. The use of the catalyst of claim 1, in the manufacture of ethylene oxide.
